(19) United States
(12) Patent Application Publication
(10) Pub. No.: US 2004/0170625 A1
(43) Pub. Date: Sep. 2, 2004

(54) Methods of treating Epidermolysis Bullosa and associated dermatological indications with thymosin beta 4, analogues, isoforms and other derivatives

(75) Inventors: Allan L. Goldstein, Washington, DC (US); Jack Finkelstein JR., Chevy Chase, MD (US)

Correspondence Address: ROTHWELL, FIGG, ERNST & MANBECK, P.C., 1425 K STREET, N.W., SUITE 800, WASHINGTON, DC, 20005, US

(73) Assignee: Regenerx Biopharmaceuticals, Inc., Bethesda, MD

(21) Appl. No.: 10/714,405

(22) Filed: Nov. 17, 2003

Related U.S. Application Data
(63) Continuation-in-part of application No. PCT/US02/15394, filed on May 16, 2002.
(63) Continuation-in-part of application No. 09/772,445, filed on Jan. 29, 2001.
Application No. 09/772,445 is a continuation of application No. PCT/US99/17282, filed on Jul. 29, 1999.
(60) Provisional application No. 60/291,326, filed on May 17, 2001.
(60) Provisional application No. 60/094,690, filed on Jul. 30, 1998.

(51) Int. Cl. A61K 39/395

(52) U.S. Cl. 424/143.1

(57) ABSTRACT

Blisters, sores or skin degradation associated with Epidermolysis Bullosa is treated or prevented by administration of an actin-sequestering peptide such as Thymosin β4, an isoform of Thymosin β4 or oxidized Thymosin β4.

### CROSS-REFERENCE TO RELATED APPLICATIONS

The present application is a continuation-in-part of PCT/US02/15394, filed May 16, 2002, which claims the benefit of U.S. Provisional Application Serial No. 60/291,326, filed May 17, 2001; and the present application is a continuation-in-part of U.S. Ser. No. 09/772,445, filed Jan. 29, 2001, which is a continuation of PCT/US99/17282, filed Jul. 29, 1999, which claims the benefit of U.S. Provisional Application Serial No. 60/094,690, filed Jul. 30,1998.

### BACKGROUND OF THE INVENTION

1. Field of the Invention

The present invention relates to the field of healing or preventing inflammatory degenerative, immunological and other disorders of the skin and surrounding tissue that occur due to Epidermolysis Bullosa, and all of its subtypes.

2. Description of the Background Art

The phenomenon called Epidermolysis Bullosa (EB) is a rare genetic disorder that afflicts all ethnic and racial groups. EB is a group of diseases characterized by blister formation after minor trauma to the skin. This may lead to open sores, ulcerations and scars. This family of disorders range in severity from mild to the severely disabling, mutilating and life threatening diseases of the skin. Unlike burns, these afflictions sometimes never go away. The most severe cases require great lifestyle adjustments. Afflicted children should never ride a bike, skate, or participate in sports, because the normal play of children causes chronic sores. Such sores may cover as much as 75 percent of the child's body. Blistering and scarring also occur in the mouth and esophagus. Therefore, frequently a diet of only liquids or soft foods is possible. Scarring also causes the fingers and toes to fuse, leaving deformities which severely limit function. Much of their life is tied to hospitals for treatment, blood transfusions, biopsies and surgeries. The eyes often blister preventing sight for days. Chronic anemia reduces energy, and growth is retarded. The life span for an individual afflicted with EB is usually not longer than 30 years.

There are three main types of EB: EB Simplex, Dystrophic EB (dominant or recessive) and Junctional EB. The severity of symptoms varies between these types. In general terms, EB causes blisters which may be restricted to specific areas, for example hands or feet, or may affect large areas of the body. In the milder forms the blisters heal normally without leaving permanent damage to the skin. In the other forms, the blisters heal with scarring which can result in permanent change to the skin, for example fingers may fuse and hands contract, reducing movement. Some forms of Junctional EB are life threatening in infancy.

EB results from deleterious changes in the physiological, biochemical and immunological properties of the skin. All forms of EB are genetic in origin and the genes responsible for several different subtypes of the condition are now known. The genetic defects result in the skin layers not adhering properly to each other, causing areas of structural weakness. This fragile skin is particularly vulnerable to damage from mild friction, causing the blisters which are the characteristic feature of the condition. Skin is an important barrier to infection, it is the first line of defense of the immune system. The fragile skin of those afflicted with EB loses this important defense mechanism. Such changes in vasculature decrease capacity to repair damage, increase propensity for skin cancers such as squamous cell carcinoma, and increase risk of infection. In addition, the open sores in the oral and digestive cavity can lead to increased dehydration and malnutrition.

There have been many attempts to treat EB, but none have had a substantial impact on prevention or treatment of EB. Various growth factors, synthetic skins, antibiotics and other therapies have failed to adequately and effectively treat EB. While EB is a genetic disease, treatment that would more rapidly heal or better heal the sores would be extremely important. Further, preventative therapy would clearly confer substantial, perhaps life-saving benefit to the patient.

Numerous pharmaceutical, nutriceutical or cosmeceutical formulations have been proposed to reduce or reverse EB or its affects.

There remains a need in the art for improved methods and compositions for healing or preventing the blisters and sores associated with EB.

### SUMMARY OF THE INVENTION

In accordance with the present invention, a method of treatment for promoting healing or prevention of blisters, sores or skin degeneration associated with EB involves administration to a subject or patient in need of such treatment an effective amount of a composition comprising an EB-inhibiting polypeptide comprising amino acid sequence LKKTET or a conservative variant thereof having EB-inhibiting activity.

### DETAILED DESCRIPTION OF THE INVENTION

The present invention is based on a discovery that actin-sequestering peptides such as thymosin β4 (Tβ4) and other actin-sequestering peptides or peptide fragments containing amino acid sequence LKKTET or conservative variants thereof, promote healing or prevention of blisters, sores and skin degeneration associated with Epidermolysis Bullosa. Without being bond to any particular theory, these peptides may have the capacity to promote repair, healing and prevention by having the ability to induce terminal deoxynucleotidyl transferase (a non-template directed DNA polymerase), to decrease the levels of one or more inflammatory cytokines and to act as a chemotactic and/or angiogenic factor for endothelial cells and thus heal and prevent degenerative changes in the skin of patients with afflicted EB, even though EB is the result of an inherited defect.

Thymosin β4 was initially identified as a protein that is up-regulated during endothelial cell migration and differentiation in vitro. Thymosin 4 was originally isolated from the thymus and is a 43 amino acid, 4.9 kDa ubiquitous polypeptide identified in a variety of tissues. Several roles have been ascribed to this protein including a role in a endothelial cell differentiation and migration, T cell differentiation, actin sequestration and vascularization.

In accordance with one embodiment, the invention is a method of treatment for promoting healing and prevention of blisters, sores and skin degradation associated with EB comprising administering to a subject in need of such treatment an effective amount of a composition comprising an EB-inhibiting peptide comprising amino acid sequence LKKTET, or a conservative variant thereof having EB-inhibiting activity, preferably Thymosin 4, an isoform of Thymosin 4, oxidized Thymosin 4 or an antagonist of Thymosin 4.

Compositions which may be used in accordance with the present invention include Thymosin 4 (T4), T4 isoforms, oxidized T4, polypeptides or peptide fragments comprising or consisting essentially of the amino acid sequence LKKTET or conservative variants thereof, having EB-inhibiting activity. International Application Serial No. PCT/US99/17282, incorporated herein by reference, discloses isoforms of T4 which may be useful in accordance with the present invention as well as amino acid sequence LKKTET and conservative variants thereof having EB-inhibiting activity, which may be utilized with the present invention. International Application Serial No. PCT/GB99/00833 (WO 99/49883), incorporated herein by reference, discloses oxidized Thymosin 4 which may be utilized in accordance with the present invention. Although the present invention is described primarily hereinafter with respect to T4 and T4 isoforms, it is to be understood that the following description is intended to be equally applicable to amino acid sequence LKKTET, peptides and fragments comprising or consisting essentially of LKKTET, conservative variants thereof having EB-inhibiting activity, as well as oxidized Thymosin 4.

In one embodiment, the invention provides a method for healing and preventing blisters and sores of skin in a subject by contacting the skin with an EB-inhibiting effective amount of a composition which contains T4 or a T4 isoform. The contacting may be topically or systemically. Examples of topical administration include, for example, contacting the skin with a lotion, salve, gel, cream, paste, spray, suspension, dispersion, hydrogel, ointment, or oil comprising T4. Systemic administration includes, for example, intravenous, intraperitoneal, intramuscular injections of a composition containing T4 or a T4 isoform. A subject may be a mammal, preferably human.

T4, or its analogues, isoforms or derivatives, may be administered in any suitable EB-inhibiting amount. For example, T4 may be administered in dosages within the range of about 0.1-50 micrograms of T4, more preferably in amounts of about 1-25 micrograms.

A composition in accordance with the present invention can be administered daily, every other day, etc., with a single application or multiple applications per day of administration, such as applications 2, 3, 4 or more times per day of administration.

T4 isoforms have been identified and have about 70%, or about 75%, or about 80% or more homology to the known amino acid sequence of T4. Such isoforms include, for example, t4ala, T9, T10, T11, T12, T13, T14 and T15. Similar to T4, the T10 and T15 isoforms have been shown to sequester actin. T4, T10 and T15, as well as these other isoforms share an amino acid sequence, LKKTET, that appears to be involved in mediating actin sequestration or binding. Although not wishing to be bound to any particular theory, the activity of T4 isoforms may be due, in part, to the ability to polymerize actin. For example, T4 can modulate actin polymerization in skin (e.g. -thymosins appear to depolymerize F-actin by sequestering free G-actin). T4's ability to modulate actin polymerization may therefore be due to all, or in part, its ability to bind to or sequester actin via the LKKTET sequence. Thus, as with T4, other proteins which bind or sequester actin, or modulate actin polymerization, including T4 isoforms having the amino acid sequence LKKTET, are likely to reduce EB, alone or in a combination with T4, as set forth herein.

Thus, it is specifically contemplated that known T4 isoforms, such as T4ala, T9, T10, T11, T12, T13, T14 and T15, as well as T4 isoforms not yet identified, will be useful in the methods of the invention. As such T4 isoforms are useful in the methods of the invention, including the methods practiced in a subject. The invention therefore further provides pharmaceutical compositions comprising T4, as well as T4 isoforms T4ala, T9, T10, T11, T12, T13, T14 and T15, and a pharmaceutically acceptable carrier.

In addition, other proteins having actin sequestering or binding capability, or that can mobilize actin or modulate actin polymerization, as demonstrated in an appropriate sequestering, binding, mobilization or polymerization assay, or identified by the presence of an amino acid sequence that mediates actin binding, such as LKKTET, for example, can similarly be employed in the methods of the invention. Such proteins include gelsolin, vitamin D binding protein (DBP), profilin, cofilin, depactin, Dnasel, vilin, fragmin, severin, capping protein, -actinin and acumentin, for example. As such methods include those practiced in a subject, the invention further provides pharmaceutical compositions comprising gelsolin, vitamin D binding protein (DBP), profilin, cofilin, depactin, Dnasel, vilin, fragmin, severin, capping protein, -actinin and acumentin as set forth herein. Thus, the invention includes the use of an EB-inhibiting polypeptide comprising the amino acid sequence LKKTET and conservative variants thereof.

As used herein, the term “conservative variant” or grammatical variations thereof denotes the replacement of an amino acid residue by another, biologically similar residue. Examples of conservative variations include the replacement of a hydrophobic residue such as isoleucine, valine, leucine or methionine for another, the replacement of a polar residue for another, such as the substitution of arginine for lysine, glutamic for aspartic acids, or glutamine for asparagine, and the like.

T4 has been localized to a number of tissue and cell types and thus, agents which stimulate the production of T4 can be added to or comprise a composition to effect T4 production from a tissue and/or a cell. Such agents include members of the family of growth factors, such as insulin-like growth factor (IGF-1), platelet derived growth factor (PDGF), epidermal growth factor (EGF), transforming growth factor beta (TGF-), basic fibroblast growth factor (bFGF), thymosin 1 (T1) and vascular endothelial growth factor (VEGF). More preferably, the agent is transforming growth factor beta (TGF-) or other members of the TGF-superfamily. T4 compositions of the invention may reduce the affects of EB by effectuating growth of the connective tissue through extracellular matrix deposition, cellular migration and vascularization of the skin.

In accordance with one embodiment, subjects are treated with an agent that stimulates production in the subject of an EB-inhibiting peptide as defined above.

Additionally, agents that assist or stimulate EB reduction may be added to a composition along with T4 or a T4 isoform. Such agents include angiogenic agents, growth factors, agents that direct differentiation of cells, agents that promote migration of cells and agents that stimulate the provision of extracellular matrix material in the skin. For example, and not by way of limitation, T4 or a T4 isoform alone or in combination can be added in combination with any one or more of the following agents: VEGF, KGF, FGF, PDGF, TGF, IGF-1, IGF-2, IL-1, prothymosin and thymosin 1 in an effective amount.

The invention also includes a pharmaceutical composition comprising a therapeutically effective amount of T4 or a T4 isoform in a pharmaceutically acceptable carrier. Such carriers include those listed above with reference to parenteral administration.

The actual dosage or reagent, formulation or composition that heals or prevents blisters, sores and skin degeneration associated with EB may depend on many factors, including the size and health of a subject. However, persons of ordinary skill in the art can use teachings describing the methods and techniques for determining clinical dosages as disclosed in PCT/US99/17282, supra, and the references cited therein, to determine the appropriate dosage to use.

Suitable topical formulations include T4 or a T4 isoform at a concentration within the range of about 0.001-10% by weight, more preferably within the range of about 0.01-0.1% by weight, most preferably about 0.05% by weight.

The therapeutic approaches described herein involve various routes of administration or delivery of reagents or compositions comprising the T4 or other compounds of the invention, including any conventional administration techniques (for example, but not limited to, topical administration, local injection, inhalation, or systemic administration), to a subject. The methods and compositions using or containing T4 or other compounds of the invention may be formulated into pharmaceutical compositions by admixture with pharmaceutically acceptable non-toxic excipients or carriers.

The invention includes use of antibodies which interact with T4 peptide or functional fragments thereof. Antibodies which consists essentially of pooled monoclonal antibodies with different epitopic specificities, as well as distinct monoclonal antibody preparations are provided. Monoclonal antibodies are made from antigen containing fragments of the protein by methods well known to those skilled in the art as disclosed in PCT/US99/17282, supra. The term antibody as used in this invention is meant to include monoclonal and polyclonal antibodies.

In yet another embodiment, the invention provides a method of treating a subject by administering an effective amount of an agent which modulates T4 gene expression. The term “modulate” refers to inhibition or suppression of T4 expression when T4 is over expressed, and induction of expression when T4 is under expressed. The term “effective amount” means that amount of T4 agent which is effective in modulating T4 gene expression resulting in reducing the symptoms of the T4 associated EB. An agent which modulates T4 or T4 isoform gene expression may be a polynucleotide for example. The polynucleotide may be an antisense, a triplex agent, or a ribozyme. For example, an antisense directed to the structural gene region or to the promoter region of T4 may be utilized.

In another embodiment, the invention provides a method for utilizing compounds that modulate T4 activity. Compounds that affect T4 activity (e.g., antagonists and agonists) include peptides, peptidomimetics, polypeptides, chemical compounds, minerals such as zincs, and biological agents.

While not be bound to any particular theory, the present invention may promote healing or prevention of blisters, sores and skin degeneration associated with Epidermolysis Bullosa by inducing terminal deoxynucleotidyl transferase (a non-template directed DNA polymerase), to decrease the levels of one or more inflammatory cytokines, and to act as a chemotactic factor for endothelial cells, and thereby promoting healing or preventing degenerative changes in skin brought about by Epidermolysis Bullosa or other degenerative or environmental factors.

1. A method of treatment for promoting healing or prevention of blisters, sores or skin degeneration associated with Epidermolysis Bullosa, comprising administering to a subject in need of such treatment an effective amount of a composition comprising an Epidermolysis Bullosa-inhibiting polypeptide comprising amino acid sequence LKKTET, or a conservative variant thereof having Epidermolysis Bullosa-inhibiting activity.

2. The method of claim 1 wherein said polypeptide promotes a skin condition improvement including an increase in skin elasticity of said subject.

3. The method of claim 1 wherein said polypeptide comprises Thymosin β4 (Tβ4), an isoform of Tβ4 or oxidized Tβ4.

4. The method of claim 1 wherein said composition is administered systemically.

5. The method of claim 1 wherein said composition is administered topically.

6. The method of claim 5 wherein said composition is in the form of a gel, creme, paste, lotion, spray, suspension, dispersion, salve, hydrogel or ointment formulation.

7. The method of claim 1 wherein said polypeptide is recombinant or synthetic.

8. The method of claim 1 wherein said polypeptide is an antibody.

9. The method of claim 8 wherein said antibody is polyclonal or monoclonal.

10. A method of treatment for promoting healing or prevention of blisters, sores or skin degeneration associated with Epidermolysis Bullosa comprising administering to a subject in need of such treatment an effective amount of a composition comprising an agent that stimulates production of an Epidermolysis Bullosa-inhibiting polypeptide comprising amino acid sequence LKKTET, or a conservative variant thereof having Epidermolysis Bullosa-inhibiting activity.

11. The method of claim 10 wherein said polypeptide is Thymosin β4.

12. The method of claim 10 wherein said agent is an antagonist of Thymosin β4.

13. A composition for use in promoting healing or prevention of blisters, sores or skin degeneration associated with Epidermolysis Bullosa comprising an effective amount of a composition including an Epidermolysis Bullosa-inhibiting polypeptide comprising amino acid sequence LKKTET or a conservative variant thereof having Epidermolysis Bullosa-inhibiting activity.

14. The composition of claim 13 wherein said polypeptide comprises Tβ4, an isoform of Tβ4 or oxidized Tβ4.

15. The composition of claim 13, comprising a gel, creme, paste, lotion, spray, suspension, dispersion salve, hydrogel or ointment formulation.
